(19) United States
(12) Patent Application Publication
(10) Pub. No.: US 2025/0034643 A1
(43) Pub. Date: Jan. 30, 2025

(54) METHOD OF PREDICTION OF PREGNANCY COMPLICATIONS BASED ON THE EXPRESSION PROFILE OF CARDIOVASCULAR MIRNAS

(71) Applicant: GENESPECTOR S.R.O., Praha (CZ)

(72) Inventor: Ilona HROMADNIKOVA, Praha (CZ)

(21) Appl. No.: 18/717,030

(22) PCT Filed: Dec. 13, 2022
(86) PCT No.: PCT/CZ2022/050129

§ 371 Date: Jun. 6, 2024

(30) Foreign Application Priority Data
Dec. 13, 2021 (CZ) PV 2021-562

(51) Int. Cl. C12Q 1/6883

(52) U.S. Cl. CPC C12Q 1/6883; C12Q 2600/118; C12Q 2600/158; C12Q 2600/178

(57) ABSTRACT

Method of prediction of pregnancy complications, such as preeclampsia, fetal growth restriction, gestational hypertension, small for gestational age fetus, preterm birth, and also detection of chronic hypertension. Pregnant women are screened to determine the expression profile of one or more miRNAs in whole peripheral venous blood collected in the period of 10th-13th gestational week, whereas said one or more miRNAs are selected from the group miR-1-3p, miR-16-5p, miR-17-5p, miR-20a-5p, miR-20b-5p, miR-21-5p, miR-24-3p, miR-26a-5p, miR-92a-3p, miR-100-5p, miR-103a-3p, miR-126-3p, miR-130b-3p, miR-133a-3p, miR-143-3p, miR-145-5p, miR-146a-5p, miR-155-5p, miR-181a-5p, miR-195-5p, miR-210-3p, miR-221-3p, miR-342-3p, miR-499a-5p, and miR-574-3p.

### TECHNICAL FIELD

The invention relates to the field of analysis of non-coding nucleic acids and expression markers, specifically microRNAs (miRNAs), using molecular-biological methods, primarily quantitative polymerase chain reaction with reverse transcription (RT-qPCR), and their use in screening to predict the development of adverse conditions in pregnancy.

### BACKGROUND ART

During pregnancy, the mother or the fetus can experience several complications that significantly contribute to maternal and perinatal morbidity and mortality. These are mainly preeclampsia (PE), fetal growth restriction (FGR), gestational hypertension (GH), small for gestational age fetus (SGA), and preterm birth (PTB) in the form spontaneous preterm birth (SPTB) or preterm premature rupture of membranes (PPROM). Additionally, chronic hypertension (CHH) is considered a risk factor for the development of PE. It is known from scientific literature, e.g. from documents Hromadníková et al., Int. J. Mol. Sci. 2020, 21(21), p 8413 or Hromadníková et al., J. Pers. Med. 2021, 11, p 39, that in addition to an acute threat to the health or life of the mother and the fetus, these complications can cause irreversible changes in the metabolism or cardiovascular and cerebrovascular systems of the mother and her child, these changes can remain undetected and their manifestation can also occur over a period of several years. Therefore, the goal of contemporary medicine is to predict these complications in time and, ideally, to prevent their occurrence or at least to mitigate their course and effects.

Current standard in clinical practice is a predictive model based on an algorithm processing data such as demographic data about the mother, her personal and obstetric history, clinical characteristics (body mass index, mean arterial pressure), results of an ultrasound examination of the uterine artery, and measured values of serum analytes beta-hCG (free beta-subunit of human chorionic gonadotropin), PAPP-A (pregnancy-associated plasma protein A), and PIGF (placental growth factor). The examination takes place during the first trimester of pregnancy and its result are given as the probability of an occurrence of trisomy 21, trisomy 18, trisomy 13, and pregnancy complications in the form of PE and FGR. In the event of an increased probability of PE occurrence, the use of low doses of acetylsalicylic acid is subsequently prescribed. However, the drawbacks of this predictive model is the inability to predict GH, i.e. another complication mentioned above, and clinical data further show that PE, FGR, SGA, and PTB are predicted by this model with insufficient success. Clinical data show that PE is successfully predicted by this algorithm in only a third of women who will develop this complication during pregnancy, and FGR in only about a quarter of women. Therefore, the goal is to find a more reliable method capable of predicting other pregnancy complications as well.

A method connecting the above-mentioned pregnancy complications with a change in the epigenetic profile of cardiovascular microRNAs (miRNAs) in mothers and children is known from documents CZ308102B6 and CZ308178B6. miRNAs are small, non-coding RNA molecules with a length of 18-25 nucleotides that influence and regulate gene expression at the post-transcriptional level. Several tissues have a characteristic miRNA expression profile and pathological processes can change this expression profile long-term or irreversibly, which can be used in diagnostics. The mentioned documents describe postpartum and postnatal changes in the expression profile of 29 miRNAs, which affect the function of metabolism and the cardiovascular system, that are induced by pregnancy complications (GH, PE, FGR). By determining the levels of miRNA biomarkers in whole peripheral venous blood, it was possible to predict the susceptibility to cardiovascular diseases in mothers who developed pregnancy complications as well as in their children. However, since the observed changes in miRNA expression profiles were caused by passed pregnancy complications, which usually manifest themselves only in the third trimester of pregnancy, i.e. not earlier than after the 20th gestational week, it is not possible to use the knowledge known from the cited documents for a development of a method that would predict complications already during the first trimester of pregnancy, i.e. in the period of 10th-13th gestational week. In other words, the increased or decreased values of the levels of given miRNAs after experiencing a pregnancy complication do not correspond in any way to their values before the onset of the complication and are completely independent of them. As the technology described in the cited patent documents is based on changes caused by the onset and progression of given pregnancy complications, using these changes to predict these complications before they occur is not possible.

It is known from scientific literature, e.g. from documents Hromadníková et al., PLoS ONE 2015, 10(9); Zhang et al., Am. J. Obstet. Gynecol. 2010, 202, p 466; Lykoudi et al., Placenta 2018, 61, p 24 či Choi et al., Placenta 2013, 34, p 799, that there are changes in the expression profile of miRNAs in placental tissue in patients who experienced PE, FGR, or GH during pregnancy. These findings were obtained retrospectively by analyzing the collected placental tissue. Document Han et al., Bratisl. Med. J. 2017, 118(9), p 523 suggests the possibility of using miR-145 as a marker to predict the onset of PE, but they test miR-145 expression only in placental tissue after delivery. In Licini et al., Transl. Res. 2021, 228, p 13, the possibility of using miR-125b as a plasma marker to predict the onset of PE in the 12th week of pregnancy is suggested, however, their predictive model for PE is based on the combination of miR-125b, pre-pregnancy BMI, and gestational age at delivery, which is, on principle, not applicable for routine PE prediction.

Furthermore, examples of changes in levels of selected circulating miRNAs during suspected PE symptoms occurring in the period of 24th-36th gestational week were described (Munaut et al., Biomed. Rep. 2016, 5, p 686). Some circulating miRNAs have been proposed to be used as markers to predict PE, but only in its severe variant (Ura et al., Taiwan. J. Obstet. Gynecol. 2014, 53, p 232). Placental-specific circulating miRNAs, i.e. miRNAs with exclusive expression in placental tissue, have been proposed to be used in the prediction of PE (Hromadníková et al., PLoS ONE 2017, 12 (2)) or PE, FGR, and GH (Hromadníková et al., Int. J. Mol. Sci. 2019, 20, p 2972). The cited documents describe markers circulating freely in serum or plasma or as part of plasma exosomes. The processing of plasma exosomes is a difficult and laborious process that is not suitable for wider use in clinical practice.

Goal of the present invention is to eliminate the drawbacks of the prior art by developing a method that is easily usable on a large scale even in a commonly equipped molecular-genetics laboratory and which is able to predict in a robust manner and with high reliability not only the frequently discussed PE and FGR, but also GH, SGA, and PTB, for which there are only limited possibilities of prediction in current clinical practice, and additionally to detect CHH as a risk factor that may potentially go undetected in many women.

### SUMMARY OF INVENTION

The present invention is based on determining levels of 25 specific miRNAs (miR-1-3p, miR-16-5p, miR-17-5p, miR-20a-5p, miR-20b-5p, miR-21-5p, miR-24-3p, miR-26a-5p, miR-92a-3p, miR-100-5p, miR-103a-3p, miR-126-3p, miR-130b-3p, miR-133a-3p, miR-143-3p, miR-145-5p, miR-146a-5p, miR-155-5p, miR-181a-5p, miR-195-5p, miR-210-3p, miR-221-3p, miR-342-3p, miR-499a-5p, miR-574-3p) by an RT-qPCR method in samples of whole peripheral venous blood taken from pregnant women during the standard first-trimester screening, i.e. in the period of 10th-13th gestational week. By selecting suitable subsets from this group of miRNAs, it is possible to predict the onset of PE, FGR, GH, SGA, and PTB with high probability and reliability and to detect CHH.

Although the connection between changes in levels of the above-mentioned miRNAs and current or past pregnancy complications in the form of PE or FGR has already been presented in scientific documents, and is therefore known from the prior art, their use for prediction is not obvious or easily inferred from these documents due to the large number of described miRNA, the high variability of possible changes in expression levels, and the necessity of non-obvious selection of subsets of these markers for the prediction of individual pregnancy complications.

The method was developed based on analyses of selected samples from 12,000 women in the first trimester of pregnancy, statistical processing of the results of these analyses, and their comparison with the subsequent course of pregnancy in the monitored women.

First, the collected blood is processed into a leukocyte lysate. The contained RNA is then extracted and short RNAs are subsequently isolated. Resulting purified solution is analyzed using a two-step RT-qPCR reaction in a device maintaining ideal temperature conditions for individual steps that are repeated cyclically in the presence of standard and sequence-specific chemicals, namely miRNA-specific stem-loop RT primers, miRNA-specific forward and reverse PCR primers, and miRNA-specific MGB probes. In each step, a fluorescence signal released from the probe is measured and the cycle is usually repeated 40-45 times in total. After finishing the program, Cₜ values are read in the individual channels and the results correspond to the original number of monitored nucleic acid molecules. RNA extraction from leukocyte lysate and RT-qPCR analysis are standards in current molecular diagnostic practice. The procedures are fast, simple to perform, and allow for easy automation. Therefore, this diagnostic method is suitable for implementation in most genetic laboratories and for wide use in clinical practice.

The normal distribution of expression of selected miRNAs was determined based on a sample of women who had a physiological pregnancy without complications. These levels were subsequently compared with miRNA levels in samples obtained from women who developed any of the monitored complications during pregnancy, and the results were statistically processed using the Kruskal-Wallis test and the Mann-Whitney test. For each individual pregnancy complication, a specific set of miRNAs was selected. These miRNAs are all up-regulated before the onset of the given complication (with the exception of PTB, where all selected miRNAs are down-regulated) and their combination enables a prediction of the complication with a sufficiently high sensitivity at a sufficiently low rate of false positives. A generally accepted value is 10% false positive rate.

The chosen miRNAs suitable as biomarkers for the prediction of individual pregnancy complications and the detection of one of the risk factors for the development of pregnancy complications (CHH) are as follows:
miR-146a-5p—up-regulated for GH, CHH, PE, FGR, SGA; down-regulated for PTB
miR-181a-5p—up-regulated for GH, CHH, PE, FGR, SGA
miR-155-5p—up-regulated for CHH, PE, FGR, SGA; down-regulated for PTB
miR-20a-5p, miR-143-3p, miR-195-5p—up-regulated for CHH, PE, FGR, SGA
miR-145-5p—up-regulated for GH, PE, FGR; down-regulated for PTB
miR-20b-5p, miR-126-3p—up-regulated for CHH, PE, SGA; down-regulated for PTB
miR-16-5p—up-regulated for PE, FGR, SGA; down-regulated for PTB
miR-574-3p—up-regulated for PE, FGR; down-regulated for PB
miR-342-3p—up-regulated for FGR; down-regulated for PTB
miR-100-5p—up-regulated for FGR, SGA
miR-1-3p, miR-499a-5p—up-regulated for CHH, SGA
miR-103a-3p—up-regulated for PE, FGR
miR-21-5p—up-regulated for PE; down-regulated for PTB
miR-17-5p—up-regulated for PE
miR-24-3p, miR-26a-5p, miR-92a-3p, miR-133a-3p, miR-210-3p, miR-221-3p—down-regulated for PTB
miR-130b-3p—up-regulated for SGA

### Preeclampsia (PE)

In women who developed PE during pregnancy, monitoring the up-regulation of the following 8 miRNAs in whole peripheral venous blood collected during the first trimester proved to be the most suitable for prediction: miR-20a-5p, miR-21-5p, miR-126-3p, miR-143-3p, miR-145-5p, miR-146a-5p, miR-181a-5p, miR-574-3p. Using this method, without any additional clinical examination of the women or knowledge of their medical history, it was possible to predict 53.0% of cases at 10% false positive rate (FPR). In the same group of women, only 33.3% of cases were detected by the prior art method using the routine predictive algorithm within a computer application for obstetrics and gynecology databases based on clinical and laboratory examination and anamnesis. Thus, using the method utilizing miRNA biomarkers, it is possible to successfully predict the onset of PE with a significantly higher probability than in the case of the prior art approach. In addition, using the method utilizing the routine predictive algorithm, it is possible to predict only the early form of PE, i.e. PE occurring before the 34th week of pregnancy, while the method utilizing miRNA biomarkers was able to predict any form of PE (early PE, late PE, mild PE, and severe PE) occurring at any stage of pregnancy after the 20th gestational week.

### Fetal Growth Restriction (FGR)

In women who experienced FGR during pregnancy, monitoring the up-regulation of the following 10 miRNAs in whole peripheral venous blood collected during the first trimester proved to be the most suitable for prediction: miR-16-5p, miR-20a-5p, miR-100-5p, miR-143-3p, miR-145-5p, miR-146a-5p, miR-181a-5p, miR-195-5p, miR-342-3p, miR-574-3p. Using this method, without any additional clinical examination of the women or knowledge of their medical history, it was possible to predict 40.2% of cases at 10% FPR. In the same group of women, only 28.0% of cases were detected by the prior art method using the routine predictive algorithm within a computer application for obstetrics and gynecology databases based on clinical and laboratory examination and anamnesis. Thus, using the method utilizing miRNA biomarkers, it is possible to successfully predict FGR with a significantly higher probability than in the case of the prior art approach. In addition, using the method utilizing the routine predictive algorithm, it is possible to predict FGR occurring only before the 37th week of pregnancy, while the method utilizing miRNA biomarkers was able to predict FGR occurring at any stage of pregnancy after the 20th gestational week, including FGR occurring after the 37th week of pregnancy.

Preterm birth (PTB) in the form of spontaneous preterm birth (SPTB) or preterm premature rupture of membranes (PPROM) without any other pregnancy complication (GH, PE, FGR, or SGA)

In women who gave birth prematurely, monitoring the down-regulation of the following 12 miRNAs in whole peripheral venous blood collected during the first trimester proved to be the most suitable for prediction: miR-16-5p, miR-20b-5p, miR-21-5p, miR-24-3p, miR-26a-5p, miR-92a-3p, miR-133a-3p, miR-145-5p, miR-146a-5p, miR-155-5p, miR-210-3p, miR-342-3p. Using this method, without any additional clinical examination of the women or knowledge of their medical history, it was possible to predict 52.8% of cases at 10% FPR. It was also possible to predict cases where the preterm birth occurred without any obvious clinical causes, which is currently practically impossible in the first trimester of pregnancy. The prior art method utilizing the routine predictive algorithm is only able to predict this complication to a very limited extent, and moreover only in the case of SPTB (20% of primiparous women, 30% of multiparous women). In addition, using the method utilizing the routine predictive algorithm, it is possible to predict only the early form of SPTB, i.e. SPTB occurring before the 34th week of pregnancy, while the method utilizing miRNA biomarkers was able to predict both SPTB and PPROM occurring at any stage of pregnancy after the 24th gestational week.

### Chronic Hypertension (CHH)

In women with chronic hypertension, which itself is a risk factor for the development of PE, the following 4 miRNAs were frequently up-regulated in whole peripheral venous blood collected during the first trimester: miR-1-3p, miR-20a-5p, miR-126-3p, miR-146a-5p. Using this method, without any additional clinical examination of the women or knowledge of their medical history, it was possible to discover 79.3% of cases at 10% FPR. The prior art method utilizing the routine predictive algorithm within a computer application for obstetrics and gynecology databases is based, among other things, on the patient's anamnesis, where CHH is part of the input as one of the risk factors in assessing the risk of developing PE. If the patient does not provide this anamnestic data during the collection of the anamnesis, it is not included in the subsequent calculation of the risk of developing PE.

### Small for Gestational Age Fetus (SGA)

In women whose fetus experienced SGA during pregnancy, monitoring the up-regulation of the following 8 miRNAs in whole peripheral venous blood collected during the first trimester proved to be the most suitable for prediction: miR-1-3p, miR-20a-5p, miR-20b-5p, miR-126-3p, miR-130b-3p, miR-146a-5p, miR-181a-5p, miR-499a-5p. Using this method, without any additional clinical examination of the women or knowledge of their medical history, it was possible to predict 83.8% of cases at 10% FPR. The prior art method utilizing the routine predictive algorithm within a computer application for obstetrics and gynecology databases is not able to predict this complication.

### Gestational Hypertension (GH)

In women who experienced GH during pregnancy, monitoring the up-regulation of the following miRNA in whole peripheral venous blood collected during the first trimester proved to be the most suitable for prediction: miR-181a-5p. Using this method, without any additional clinical examination of the women or knowledge of their medical history, it was possible to predict 22.9% of cases at 10% FPR. The prior art method utilizing the routine predictive algorithm within a computer application for obstetrics and gynecology databases is not able to predict this complication.

### BRIEF DESCRIPTION OF DRAWINGS

FIG. 1 depicts ROC (Receiver Operating Characteristic) curves obtained from the statistical analysis of the up-regulation of 8 selected miRNAs (miR-20a-5p, miR-21-5p, miR-126-3p, miR-143-3p, miR-145-5p, miR-146a-5p, miR-181a-5p, miR-574-3p) for prediction of PE, both for combined screening and for individual miRNAs.

FIG. 2 depicts ROC curves obtained from the statistical analysis of the up-regulation of 10 selected miRNAs (miR-16-5p, miR-20a-5p, miR-100-5p, miR-143-3p, miR-145-5p, miR-146a-5p, miR-181a-5p, miR-195-5p, miR-342-3p, miR-574-3p) for prediction of FGR, both for combined screening and for individual miRNAs.

FIG. 3 depicts ROC curves obtained from the statistical analysis of the down-regulation of 12 selected miRNAs (miR-16-5p, miR-20b-5p, miR-21-5p, miR-24-3p, miR-26a-5p, miR-92a-3p, miR-133a-3p, miR-145-5p, miR-146a-5p, miR-155-5p, miR-210-3p, miR-342-3p) for prediction of PTB in the form of SPTB or PPROM without any other pregnancy complication (GH, PE, FGR, or SGA), both for combined screening and for individual miRNAs.

FIG. 4 depicts ROC curves obtained from the statistical analysis of the up-regulation of 4 selected miRNAs (miR-1-3p, miR-20a-5p, miR-126-3p, miR-146a-5p) for detection of CHH, both for combined screening and for individual miRNAs.

FIG. 5 depicts ROC curves obtained from the statistical analysis of the up-regulation of 8 selected miRNAs (miR-1-3p, miR-20a-5p, miR-20b-5p, miR-126-3p, miR-130b-3p, miR-146a-5p, miR-181a-5p, miR-499a-5p) for prediction of SGA, both for combined screening and for individual miRNAs.

FIG. 6 depicts ROC curve obtained from the statistical analysis of the up-regulation of a selected miRNA (miR-181a-5p) for prediction of GH.

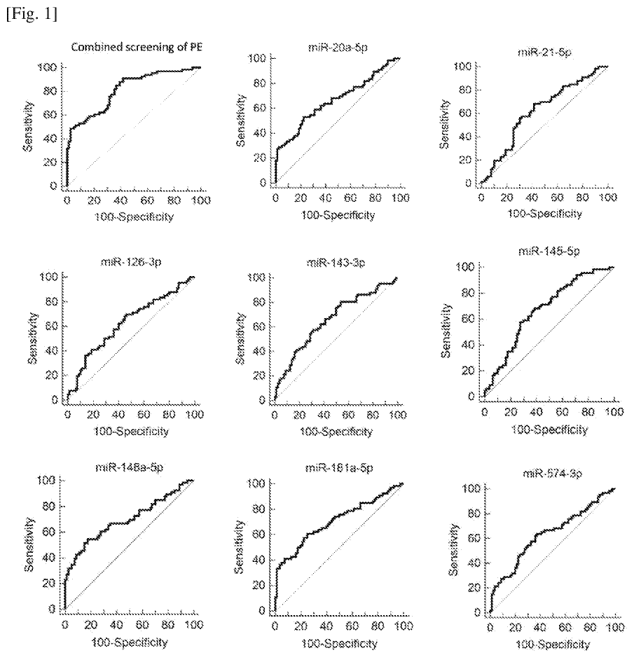

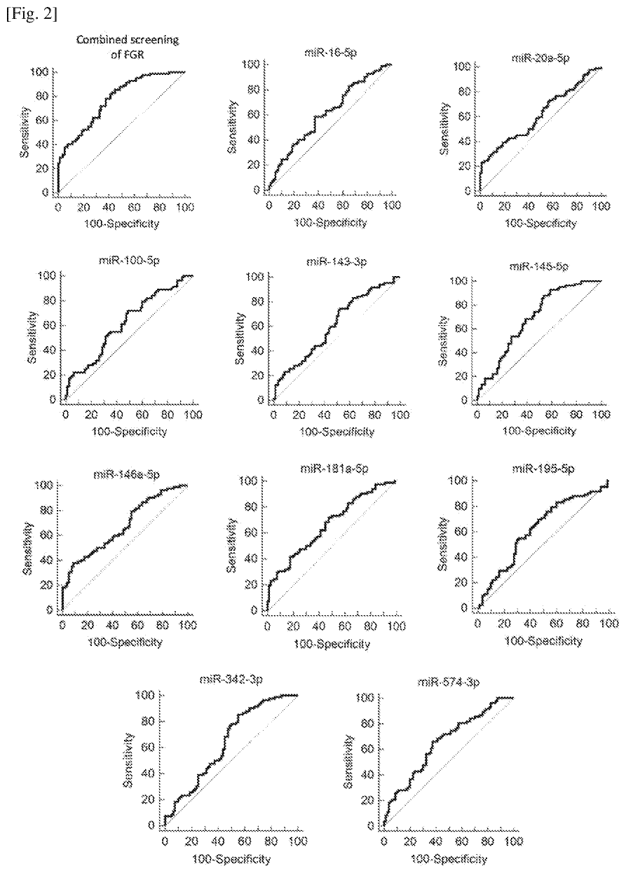

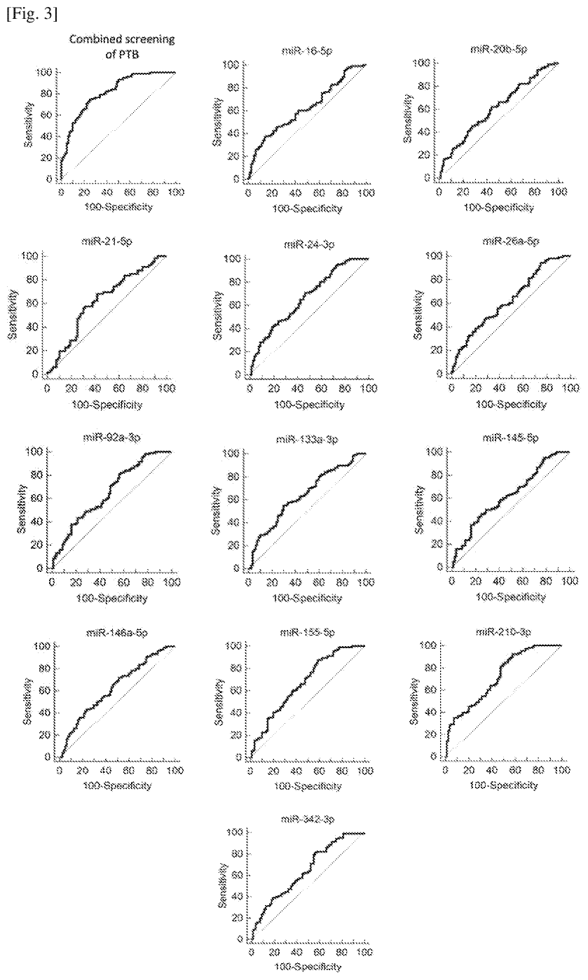

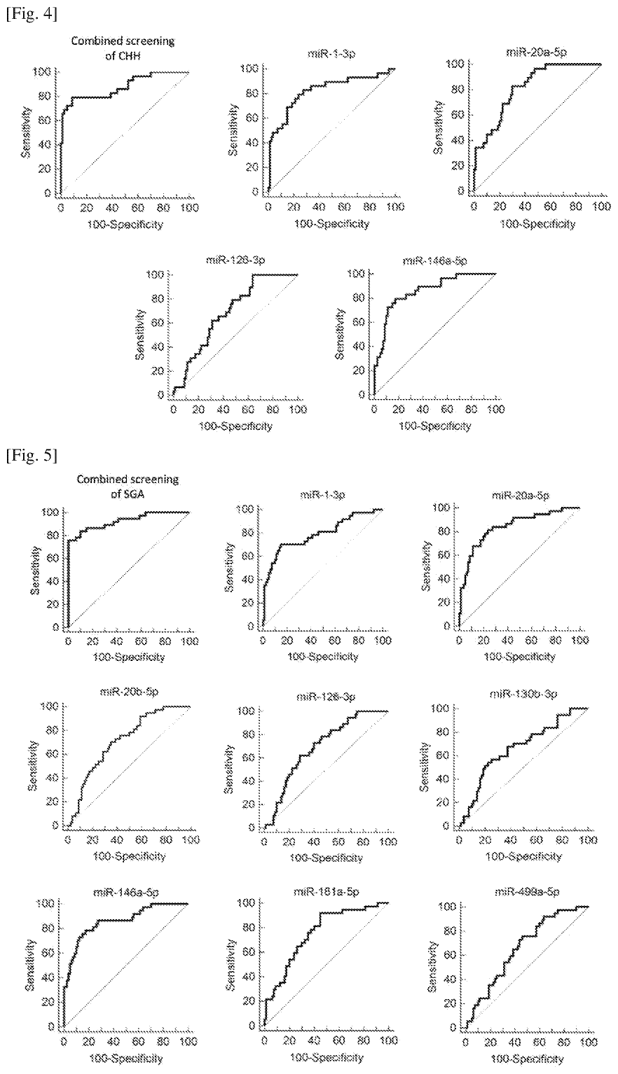

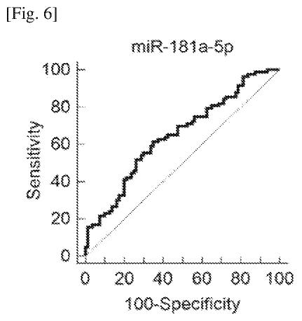

### DESCRIPTION OF EMBODIMENTS

### Example 1

Example 1 describes a general implementation of RT-qPCR analysis determining the amount of miRNA in a tested sample of whole peripheral venous blood and in a reference sample normalized to a simultaneously determined amount of selected endogenous controls (RNU58A a RNU38B).

RNA isolation is performed from thawed leukocyte lysate using a mixture of acidic phenol and chloroform. Long RNAs are further removed from the obtained RNA and short RNAs are concentrated using a column with a glass fiber filter and ethanol of different concentrations in the individual isolation steps. The isolated RNA containing short RNAs is directly used as a template in a two-step RT-qPCR reaction. Reverse transcription takes place under the following conditions: 30 min at 16° C., 30 min at 42° C., and 5 min at 85° C. This is followed by a polymerase chain reaction under the following conditions: 95° C. for 10 min, 95° C. for 15 s, and 60° C. for 1 min. After this step, the fluorescence is measured every time in the FAM and ROX channels (passive reference for fluorescence normalization). This cycle is repeated a total of 40 to 45 times. After the program ends, Cₜ values in the individual channels are read. Within one sample, the Cₜ value in the FAM channel is obtained for the measured miRNA or for the short RNA serving as an endogenous control. These values correspond to the expression of individual genes in the biological sample. The normalized expression value is obtained by subtracting the Cₜ value of the endogenous control (geometric mean of RNU58A and RNU38B) from the Ct value of the miRNA in the assayed sample. For relative quantification, the expression of all studied miRNAs and endogenous controls is determined simultaneously also in a reference sample, which is used in all performed analyses.

### Example 2

Example 2 describes a general implementation of statistical analysis of data describing the level of selected miRNAs.

Due to the non-normal distribution of data according to the Shapiro-Wilk test, non-parametric tests are used to evaluate the experimental data. Gene expression of miRNAs is compared between individual groups using the Mann-Whitney test and in case of more than 2 compared groups using the Kruskal-Wallis test followed by a post-hoc analysis. The level of statistical significance is set at a value of p<0.05. ROC (Receiver Operating Characteristic) curves are also constructed for the respective miRNAs. The area under the curve, the sensitivity and specificity of individual miRNAs, and the optimal cut-off value (the so-called criterion) are evaluated. Furthermore, the optimal cut-off value and sensitivity of a given miRNA biomarker is determined at 90.0% specificity, which corresponds to information about the percentage of women with increased or decreased expression of a specific miRNA at 10.0% false positive rate (FPR). Furthermore, a combined statistical analysis in the form of logistic regression and ROC analysis is performed in order to select the optimal combination of miRNA biomarkers for the given situation. This application provides the following parameters: area under the curve, sensitivity, specificity, optimal cut-off value, and sensitivity of a given combination of miRNA biomarkers at 90.0% specificity.

### Example 3

Example 3 describes a collection and selection of a suitable set of biological samples for the development of a method for predicting pregnancy complications using cardiovascular miRNAs as biomarkers.

200 μL of whole peripheral venous blood is collected from approximately 12 thousand women in 10th-13th week of pregnancy. A cell lysate of leukocytes is prepared by removing erythrocytes and then stored deep-frozen at −80° C. After the patients give birth, samples from women whose complete state of health during the entire pregnancy is known, including all complications and the course of childbirth, are specifically selected. 80 patients with a physiological course of pregnancy, i.e. without any complications, and with a negative result of the first-trimester prenatal screening using a prior art method utilizing the routine predictive algorithm within a computer application for obstetrics and gynecology databases are selected as a control group. For the study of individual complications, the following patient groups are selected: 66 patients who were diagnosed with PE during pregnancy, 82 patients who were diagnosed with FGR during pregnancy, 106 patients who gave birth prematurely (SPTB or PPROM) and at the same time experienced no other complications (GH, PE, FGR, or SGA), 29 patients with chronic hypertension, 37 patients who were diagnosed with SGA during pregnancy, and 83 patients who were diagnosed with GH during pregnancy. Blood samples of these selected patients are analyzed following the procedure described in Example 1 and levels of selected 25 miRNAs are determined (miR-1-3p, miR-16-5p, miR-17-5p, miR-20a-5p, miR-20b-5p, miR-21-5p, miR-24-3p, miR-26a-5p, miR-92a-3p, miR-100-5p, miR-103a-3p, miR-126-3p, miR-130b-3p, miR-133a-3p, miR-143-3p, miR-145-5p, miR-146a-5p, miR-155-5p, miR-181a-5p, miR-195-5p, miR-210-3p, miR-221-3p, miR-342-3p, miR-499a-5p, miR-574-3p). For the prediction of individual pregnancy complications with sufficient reliability described in Examples 4-9, more than half of the suitable miRNAs shown in the individual examples are used each time. The highest reliability is achieved by using all suitable miRNAs shown in the individual examples.

### Example 4

Example 4 describes a successful prediction of PE using selected miRNA markers in a selected sample of patients.

66 monitored patients develop PE during pregnancy, with 17 patients having a mild form and 49 patients having a severe form of PE. Of the monitored patients, 14 give birth before the 34th gestational week (early PE) and 52 give birth after the 34th gestational week (late PE). Blood samples of these selected patients are analyzed following the procedure described in Example 1 and levels of selected 8 miRNAs are determined (miR-20a-5p, miR-21-5p, miR-126-3p, miR-143-3p, miR-145-5p, miR-146a-5p, miR-181a-5p, miR-574-3p). Up-regulation of these selected miRNA biomarkers, whose levels exceed the minimum values determined by a statistical analysis for a 10% FPR, is observed in 35 of 66 patients (9 mild PE, 26 severe PE, 5 early PE, 30 late PE), which corresponds to a successful prediction in 53.0% of cases. Statistical analysis of the obtained data provides the following values of specificity, sensitivity, 95% CI, and criterion:

Combined screening of PE (8 selected miRNA)

| Area Under the ROC Curve | 0.815 | Criterion | >0.284019788 |
|---|---|---|---|
| (AUC) | | | |
| Standard Error | 0.0351 | Sensitivity | 90.91 |
| 95% Confidence Interval | 0.742-0.875 | 95% CI | 81.3-96.6 |
| Significance Level P | <0.0001 | Specificity | 58.23 |
| (Area = 0.5) | | | |
| Estimated sensitivity at fixed specificity | | 95% CI | 46.6-69.2 |
| Specificity | 90.00 | +LR | 2.18 |
| Sensitivity | 53.03 | 95% CI | 1.7-2.9 |
| 95% Confidence Interval | 39.39-65.15 | −LR | 0.16 |
| Criterion | >0.54823634 | 95% CI | 0.07-0.3 |

miR-20a-5p

| Area Under the ROC Curve | 0.666 | Criterion | >2.43637 |
|---|---|---|---|
| (AUC) | | | |
| Standard Error | 0.0459 | Sensitivity | 53.03 |
| 95% Confidence Interval | 0.584-0.742 | 95% CI | 40.3-65.4 |
| Significance Level P | 0.0003 | Specificity | 77.50 |
| (Area = 0.5) | | | |
| Estimated sensitivity at fixed specificity | | 95% CI | 66.8-86.1 |
| Specificity | 90.00 | +LR | 2.36 |
| Sensitivity | 33.33 | 95% CI | 1.5-3.8 |
| 95% Confidence Interval | 22.73-46.97 | LR | 0.61 |
| Criterion | >3.587078 | 95% CI | 0.5-0.8 |

miR-21a-5p

| Area Under the ROC Curve | 0.624 | Criterion | >0.350516 |
|---|---|---|---|
| (AUC) | | | |
| Standard Error | 0.0468 | Sensitivity | 68.18 |
| 95% Confidence Interval | 0.539-0.703 | 95% CI | 55.6-79.1 |
| Significance Level P | 0.0083 | Specificity | 58.23 |
| (Area = 0.5) | | | |
| Estimated sensitivity at fixed specificity | | 95% CI | 46.6-69.2 |
| Specificity | 90.00 | +LR | 1.63 |
| Sensitivity | 13.64 | 95% CI | 1.2-2.2 |
| 95% Confidence Interval | 1.52-27.27 | −LR | 0.55 |
| Criterion | >0.7863184 | 95% CI | 0.4-0.8 |

miR-126-3p

| Area Under the ROC Curve | 0.629 | Criterion | >0.282106 |
|---|---|---|---|
| (AUC) | | | |
| Standard Error | 0.0468 | Sensitivity | 69.70 |
| 95% Confidence Interval | 0.545-0.708 | 95% CI | 57.1-80.4 |
| Significance Level P | 0.0058 | Specificity | 53.75 |
| (Area = 0.5) | | | |
| Estimated sensitivity at fixed specificity | | 95% CI | 42.2-65.0 |
| Specificity | 90.00 | +LR | 1.51 |
| Sensitivity | 24.24 | 95% CI | 1.1-2.0 |
| 95% Confidence Interval | 6.06-45.45 | −LR | 0.56 |
| Criterion | >0.659038 | 95% CI | 0.4-0.9 |

miR-143-3p

| Area Under the ROC Curve | 0.659 | Criterion | >0.031066 |
|---|---|---|---|
| (AUC) | | | |
| Standard Error | 0.0456 | Sensitivity | 80.30 |
| 95% Confidence Interval | 0.576-0.736 | 95% CI | 68.7-89.1 |
| Significance Level P | 0.0005 | Specificity | 46.25 |
| (Area = 0.5) | | | |
| Estimated sensitivity at fixed specificity | | 95% CI | 35.0-57.8 |
| Specificity | 90.00 | +LR | 1.49 |
| Sensitivity | 24.24 | 95% CI | 1.2-1.9 |
| 95% Confidence Interval | 12.12-42.42 | −LR | 0.43 |
| Criterion | >0.129856 | 95% CI | 0.2-0.7 |

miR-145-5p

| Area Under the ROC Curve | 0.673 | Criterion | >0.223013 |
|---|---|---|---|
| (AUC) | | | |
| Standard Error | 0.0444 | Sensitivity | 57.85 |
| 95% Confidence Interval | 0.590-0.748 | 95% CI | 44.8-69.7 |
| Significance Level P | 0.0001 | Specificity | 72.50 |
| (Area = 0.5) | | | |
| Estimated sensitivity at fixed specificity | | 95% CI | 61.4-81.9 |
| Specificity | 90.00 | +LR | 2.09 |
| Sensitivity | 21.21 | 95% CI | 1.4-3.2 |
| 95% Confidence Interval | 9.09-37.88 | −LR | 0.59 |
| Criterion | >0.403208 | 95% CI | 0.4-0.8 |

miR-146-5p

| Area Under the ROC Curve | 0.698 | Criterion | >2.073306 |
|---|---|---|---|
| (AUC) | | | |
| Standard Error | 0.0450 | Sensitivity | 54.55 |
| 95% Confidence Interval | 0.617-0.771 | 95% CI | 41.8-66.9 |
| Significance Level P | <0.0001 | Specificity | 82.50 |
| (Area = 0.5) | | | |
| Estimated sensitivity at fixed specificity | | 95% CI | 72.4-90.1 |
| Specificity | 90.00 | +LR | 3.12 |
| Sensitivity | 42.42 | 95% CI | 1.8-5.3 |
| 95% Confidence Interval | 27.27-57.58 | −LR | 0.55 |
| Criterion | >2.744534 | 95% CI | 0.4-0.7 |

miR-181a-5p

| Area Under the ROC Curve | 0.707 | Criterion | >0.32734 |
|---|---|---|---|
| (AUC) | | | |
| Standard Error | 0.0442 | Sensitivity | 60.61 |
| 95% Confidence Interval | 0.626-0.779 | 95% CI | 47.8-72.4 |
| Significance Level P | <0.0001 | Specificity | 75.00 |
| (Area = 0.5) | | | |
| Estimated sensitivity at fixed specificity | | 95% CI | 64.1-84.0 |
| Specificity | 90.00 | +LR | 2.42 |
| Sensitivity | 41.91 | 95% CI | 1.6-3.7 |
| 95% Confidence Interval | 29.54-54.55 | −LR | 0.53 |
| Criterion | >0.521629 | 95% CI | 0.4-0.7 |

miR-574a-3p

| Area Under the ROC Curve | 0.636 | Criterion | >0.202286 |
|---|---|---|---|
| (AUC) | | | |
| Standard Error | 0.0467 | Sensitivity | 63.64 |
| 95% Confidence Interval | 0.553-0.714 | 95% CI | 50.9-75.1 |
| Significance Level P | 0.0035 | Specificity | 62.50 |
| (Area = 0.5) | | | |
| Estimated sensitivity at fixed specificity | | 95% CI | 51.0-73.1 |
| Specificity | 90.00 | +LR | 1.70 |
| Sensitivity | 27.27 | 95% CI | 1.2-2.4 |
| 95% Confidence Interval | 18.18-42.42 | −LR | 0.58 |
| Criterion | >0.42623 | 95% CI | 0.4-0.8 |

CI = Confidence Interval
LR = Likelihood Ratio

### Example 5

Example 5 describes a successful prediction of FGR using selected miRNA markers in a selected sample of patients.

82 monitored patients are diagnosed with FGR during pregnancy, with 5 patients giving birth before the 32nd gestational week (early FGR) and 77 giving birth after the 32nd gestational week (late FGR). Blood samples of these selected patients are analyzed following the procedure described in Example 1 and levels of selected 10 miRNAs are determined (miR-16-5p, miR-20a-5p, miR-100-5p, miR-143-3p, miR-145-5p, miR-146a-5p, miR-181a-5p, miR-195-5p, miR-342-3p, miR-574-3p). Up-regulation of these selected miRNA biomarkers, whose levels exceed the minimum values determined by a statistical analysis for a 10% FPR, is observed in 33 of 82 patients (2 early FGR, 31 late FGR), which corresponds to a successful prediction in 40.2% of cases. Statistical analysis of the obtained data provides the following values of specificity, sensitivity, 95% CI, and criterion:

Combined screening of FGR (10 selected miRNA)

| Area Under the ROC Curve | 0.774 | Criterion | >0.393364539 |
|---|---|---|---|
| (AUC) | | | |
| Standard Error | 0.0358 | Sensitivity | 78.05 |
| 95% Confidence Interval | 0.702-0.836 | 95% CI | 67.5-86.4 |
| Significance Level P | <0.0001 | Specificity | 62.50 |
| (Area = 0.5) | | | |
| Estimated sensitivity at fixed specificity | | 95% CI | 51.0-73.1 |
| Specificity | 90.00 | +LR | 2.08 |
| Sensitivity | 40.24 | 95% CI | 1.5-2.8 |
| 95% Confidence Interval | 25.61-51.22 | −LR | 0.35 |
| Criterion | >0.658966306 | 95% CI | 0.2-0.5 |

miR-16-5p

| Area Under the ROC Curve | 0.615 | Criterion | >1.545201 |
|---|---|---|---|
| (AUC) | | | |
| Standard Error | 0.0440 | Sensitivity | 58.54 |
| 95% Confidence Interval | 0.536-0.690 | 95% CI | 47.1-69.3 |
| Significance Level P | 0.0090 | Specificity | 62.50 |
| (Area = 0.5) | | | |
| Estimated sensitivity at fixed specificity | | 95% CI | 51.0-73.1 |
| Specificity | 90.00 | +LR | 1.56 |
| Sensitivity | 24.39 | 95% CI | 1.1-2.2 |
| 95% Confidence Interval | 12.20-39.79 | −LR | 0.66 |
| Criterion | >3.02212 | 95% CI | 0.5-0.9 |

miR-20a-5p

| Area Under the ROC Curve | 0.615 | Criterion | >5.003977 |
|---|---|---|---|
| (AUC) | | | |
| Standard Error | 0.0442 | Sensitivity | 23.17 |
| 95% Confidence Interval | 0.535-0.690 | 95% CI | 14.6-33.8 |
| Significance Level P | 0.0094 | Specificity | 98.75 |
| (Area = 0.5) | | | |
| Estimated sensitivity at fixed specificity | | 95% CI | 93.2-100.0 |
| Specificity | 90.00 | +LR | 18.54 |
| Sensitivity | 30.49 | 95% CI | 2.5-135.2 |
| 95% Confidence Interval | 18.29-42.68 | −LR | 0.78 |
| Criterion | >3.587078 | 95% CI | 0.7-0.9 |

miR-100-5p

| Area Under the ROC Curve | 0.622 | Criterion | >0.001336 |
|---|---|---|---|
| (AUC) | | | |
| Standard Error | 0.0440 | Sensitivity | 71.95 |
| 95% Confidence Interval | 0.543-0.697 | 95% CI | 60.9-81.3 |
| Significance Level P | 0.0055 | Specificity | 51.25 |
| (Area = 0.5) | | | |
| Estimated sensitivity at fixed specificity | | 95% CI | 39.8-62.6 |
| Specificity | 90.00 | +LR | 1.48 |
| Sensitivity | 21.95 | 95% CI | 1.1-1.9 |
| 95% Confidence Interval | 13.41-32.93 | −LR | 0.55 |
| Criterion | >0.003876 | 95% CI | 0.4-0.8 |

miR-143-3p

| Area Under the ROC Curve | 0.610 | Criterion | >0.033436 |
|---|---|---|---|
| (AUC) | | | |
| Standard Error | 0.0443 | Sensitivity | 73.17 |
| 95% Confidence Interval | 0.531-0.686 | 95% CI | 62.2-82.4 |
| Significance Level P | 0.0127 | Specificity | 48.75 |
| (Area = 0.5) | | | |
| Estimated sensitivity at fixed specificity | | 95% CI | 37.4-60.2 |
| Specificity | 90.00 | +LR | 1.43 |
| Sensitivity | 23.17 | 95% CI | 1.1-1.8 |
| 95% Confidence Interval | 13.41-34.15 | −LR | 0.55 |
| Criterion | >0.129856 | 95% CI | 0.4-0.8 |

miR-145-5p

| Area Under the ROC Curve | 0.687 | Criterion | >0.149246 |
|---|---|---|---|
| (AUC) | | | |
| Standard Error | 0.0419 | Sensitivity | 87.80 |
| 95% Confidence Interval | 0.610-0.758 | 95% CI | 78.7-94.0 |
| Significance Level P | <0.0001 | Specificity | 46.25 |
| (Area = 0.5) | | | |
| Estimated sensitivity at fixed specificity | | 95% CI | 35.0-57.8 |
| Specificity | 90.00 | +LR | 1.63 |
| Sensitivity | 18.29 | 95% CI | 1.3-2.0 |
| 95% Confidence Interval | 8.54-30.49 | −LR | 0.26 |
| Criterion | >0.403208 | 95% CI | 0.1-0.5 |

miR-146a-5p

| Area Under the ROC Curve | 0.676 | Criterion | >2.820447 |
|---|---|---|---|
| (AUC) | | | |
| Standard Error | 0.0417 | Sensitivity | 37.80 |
| 95% Confidence Interval | 0.598-0.747 | 95% CI | 27.3-49.2 |
| Significance Level P | <0.0001 | Specificity | 91.25 |
| (Area = 0.5) | | | |
| Estimated sensitivity at fixed specificity | | 95% CI | 82.8-96.4 |
| Specificity | 90.00 | +LR | 4.32 |
| Sensitivity | 37.80 | 95% CI | 2.0-9.2 |
| 95% Confidence Interval | 24.39-51.22 | −LR | 0.68 |
| Criterion | >2.744534 | 95% CI | 0.6-0.8 |

miR-181a-5p

| Area Under the ROC Curve | 0.663 | Criterion | >0.183648 |
|---|---|---|---|
| (AUC) | | | |
| Standard Error | 0.0423 | Sensitivity | 71.95 |
| 95% Confidence Interval | 0.584-0.735 | 95% CI | 60.9-81.3 |
| Significance Level P | 0.0001 | Specificity | 52.50 |
| (Area = 0.5) | | | |
| Estimated sensitivity at fixed specificity | | 95% CI | 41.0-63.8 |
| Specificity | 90.00 | +LR | 1.51 |
| Sensitivity | 30.49 | 95% CI | 1.2-2.0 |
| 95% Confidence Interval | 19.51-44.25 | −LR | 0.53 |
| Criterion | >0.521629 | 95% CI | 0.4-0.8 |

miR-195a-5p

| Area Under the ROC Curve | 0.623 | Criterion | >0.176527 |
|---|---|---|---|
| (AUC) | | | |
| Standard Error | 0.0444 | Sensitivity | 53.66 |
| 95% Confidence Interval | 0.544-0.698 | 95% CI | 42.3-64.7 |
| Significance Level P | 0.0056 | Specificity | 70.00 |
| (Area = 0.5) | | | |
| Estimated sensitivity at fixed specificity | | 95% CI | 58.7-79.7 |
| Specificity | 90.00 | +LR | 1.79 |
| Sensitivity | 19.51 | 95% CI | 1.2-2.6 |
| 95% Confidence Interval | 7.32-32.93 | −LR | 0.66 |
| Criterion | >0.553901 | 95% CI | 0.5-0.9 |

miR-342-3p

| Area Under the ROC Curve | 0.642 | Criterion | >2.333226 |
|---|---|---|---|
| (AUC) | | | |
| Standard Error | 0.0438 | Sensitivity | 85.37 |
| 95% Confidence Interval | 0.563-0.716 | 95% CI | 75.8-92.2 |
| Significance Level P | 0.0012 | Specificity | 45.00 |
| (Area = 0.5) | | | |
| Estimated sensitivity at fixed specificity | | 95% CI | 33.8-56.5 |
| Specificity | 90.00 | +LR | 1.55 |
| Sensitivity | 20.73 | 95% CI | 1.2-1.9 |
| 95% Confidence Interval | 7.32-31.71 | −LR | 0.33 |
| Criterion | >5.904859 | 95% CI | 0.2-0.6 |

miR-574-3p

| Area Under the ROC Curve | 0.655 | Criterion | >0.202286 |
|---|---|---|---|
| (AUC) | | | |
| Standard Error | 0.0428 | Sensitivity | 65.85 |
| 95% Confidence Interval | 0.576-0.727 | 95% CI | 54.6-76.0 |
| Significance Level P | 0.0003 | Specificity | 62.50 |
| (Area = 0.5) | | | |
| Estimated sensitivity at fixed specificity | | 95% CI | 51.0-73.1 |
| Specificity | 90.00 | +LR | 1.76 |
| Sensitivity | 26.83 | 95% CI | 1.3-2.4 |
| 95% Confidence Interval | 15.85-39.16 | −LR | 0.55 |
| Criterion | >0.42623 | 95% CI | 0.4-0.8 |

CI = Confidence Interval
LR = Likelihood Ratio

### Example 6

Example 6 describes a successful prediction of PTB in the form of SPTB or PPROM without any other pregnancy complications (GH, PE, FGR, or SGA) using selected miRNA markers in a selected sample of patients.

106 monitored patients without any other pregnancy complications manifest either spontaneous preterm birth (41 patients) or preterm premature rupture of membranes (65 patients) before the 37th gestational week, with 29 patients giving birth before the 34th gestational week and 77 patients giving birth after the 34th gestational week. Blood samples of these selected patients are analyzed following the procedure described in Example 1 and levels of selected 12 miRNAs are determined (miR-16-5p, miR-20b-5p, miR-21-5p, miR-24-3p, miR-26a-5p, miR-92a-3p, miR-133a-3p, miR-145-5p, miR-146a-5p, miR-155-5p, miR-210-3p, miR-342-3p). Down-regulation of these selected miRNA biomarkers, whose levels are below the maximum values determined by a statistical analysis for a 10% FPR, is observed in 56 of 106 patients (13 SPTB, 43 PPROM, 12 PTB before 34th week, 44 PTB after 34th week), which corresponds to a successful prediction in 52.8% of cases. Statistical analysis of the obtained data provides the following values of specificity, sensitivity, 95% CI, and criterion:

Combined screening of PTB (12 selected miRNA)

| Area Under the ROC Curve | 0.818 | Criterion | >0.633939505 |
|---|---|---|---|
| (AUC) | | | |
| Standard Error | 0.0308 | Sensitivity | 74.53 |
| 95% Confidence Interval | 0.754-0.870 | 95% CI | 65.1-82.5 |
| Significance Level P | <0.0001 | Specificity | 75.00 |
| (Area = 0.5) | | | |
| Estimated sensitivity at fixed specificity | | 95% CI | 64.1-84.0 |
| Specificity | 90.00 | +LR | 2.98 |
| Sensitivity | 52.83 | 95% CI | 2.0-4.4 |
| 95% Confidence Interval | 32.08-68.87 | −LR | 0.34 |
| Criterion | >0.735198275 | 95% CI | 0.2-0.5 |

miR-16-5p

| Area Under the ROC Curve | 0.625 | Criterion | ≤0.70928317 |
|---|---|---|---|
| (AUC) | | | |
| Standard Error | 0.0409 | Sensitivity | 36.79 |
| 95% Confidence Interval | 0.551-0.695 | 95% CI | 27.6-46.7 |
| Significance Level P | 0.0022 | Specificity | 86.25 |
| (Area = 0.5) | | | |
| Estimated sensitivity at fixed specificity | | 95% CI | 76.7-92.9 |
| Specificity | 90.00 | +LR | 2.68 |
| Sensitivity | 29.25 | 95% CI | 1.5-4.9 |
| 95% Confidence Interval | 14.15-41.51 | −LR | 0.73 |
| Criterion | ≤0.597445814 | 95% CI | 0.6-0.9 |

miR-20-5p

| Area Under the ROC Curve | 0.612 | Criterion | ≤1.66069881 |
|---|---|---|---|
| (AUC) | | | |
| Standard Error | 0.0415 | Sensitivity | 62.26 |
| 95% Confidence Interval | 0.537-0.682 | 95% CI | 52.3-71.5 |
| Significance Level P | 0.0072 | Specificity | 56.25 |
| (Area = 0.5) | | | |
| Estimated sensitivity at fixed specificity | | 95% CI | 44.7-67.3 |
| Specificity | 90.00 | +LR | 1.42 |
| Sensitivity | 21.70 | 95% CI | 1.1-1.9 |
| 95% Confidence Interval | 10.38-34.91 | −LR | 0.67 |
| Criterion | ≤0.531505672 | 95% CI | 0.5-0.9 |

miR-21-5p

| Area Under the ROC Curve | 0.606 | Criterion | ≤0.115689703 |
|---|---|---|---|
| (AUC) | | | |
| Standard Error | 0.0414 | Sensitivity | 36.79 |
| 95% Confidence Interval | 0.532-0.677 | 95% CI | 27.6-46.7 |
| Significance Level P | 0.0102 | Specificity | 87.50 |
| (Area = 0.5) | | | |
| Estimated sensitivity at fixed specificity | | 95% CI | 78.2-93.8 |
| Specificity | 90.00 | +LR | 2.94 |
| Sensitivity | 33.02 | 95% CI | 1.6-5.5 |
| 95% Confidence Interval | 16.04-47.17 | −LR | 0.72 |
| Criterion | ≤0.100086787 | 95% CI | 0.6-0.9 |

miR-24-3p

| Area Under the ROC Curve | 0.666 | Criterion | ≤0.319091663 |
|---|---|---|---|
| (AUC) | | | |
| Standard Error | 0.0400 | Sensitivity | 70.75 |
| 95% Confidence Interval | 0.593-0.733 | 95% CI | 61.1-79.2 |
| Significance Level P | <0.0001 | Specificity | 53.75 |
| (Area = 0.5) | | | |
| Estimated sensitivity at fixed specificity | | 95% CI | 42.2-65.0 |
| Specificity | 90.00 | +LR | 1.53 |
| Sensitivity | 28.30 | 95% CI | 1.2-2.0 |
| 95% Confidence Interval | 13.21-41.51 | LR | 0.54 |
| Criterion | ≤0.107445 | 95% CI | 0.4-0.8 |

miR-26a-5p

| Area Under the ROC Curve | 0.630 | Criterion | ≤1.076532768 |
|---|---|---|---|
| (AUC) | | | |
| Standard Error | 0.0411 | Sensitivity | 94.34 |
| 95% Confidence Interval | 0.556-0.699 | 95% CI | 88.1-97.9 |
| Significance Level P | 0.0016 | Specificity | 25.00 |
| (Area = 0.5) | | | |
| Estimated sensitivity at fixed specificity | | 95% CI | 16.0-35.9 |
| Specificity | 90.00 | +LR | 1.26 |
| Sensitivity | 23.58 | 95% CI | 1.1-1.4 |
| 95% Confidence Interval | 11.64-37.74 | −LR | 0.23 |
| Criterion | ≤0.210206711 | 95% CI | 0.10-0.5 |

miR-92a-3p

| Area Under the ROC Curve | 0.653 | Criterion | ≤2.777040345 |
|---|---|---|---|
| (AUC) | | | |
| Standard Error | 0.0407 | Sensitivity | 81.13 |
| 95% Confidence Interval | 0.580-0.721 | 95% CI | 72.4-88.1 |
| Significance Level P | 0.0002 | Specificity | 43.75 |
| (Area = 0.5) | | | |
| Estimated sensitivity at fixed specificity | | 95% CI | 32.7-55.3 |
| Specificity | 90.00 | +LR | 1.44 |
| Sensitivity | 22.64 | 95% CI | 1.2-1.8 |
| 95% Confidence Interval | 9.43-38.68 | −LR | 0.43 |
| Criterion | ≤0.644813403 | 95% CI | 0.3-0.7 |

miR-133a-3p

| Area Under the ROC Curve | 0.639 | Criterion | ≤0.065504237 |
|---|---|---|---|
| (AUC) | | | |
| Standard Error | 0.0408 | Sensitivity | 54.72 |
| 95% Confidence Interval | 0.566-0.708 | 95% CI | 44.8-64.4 |
| Significance Level P | 0.0006 | Specificity | 70.00 |
| (Area = 0.5) | | | |
| Estimated sensitivity at fixed specificity | | 95% CI | 58.7-79.7 |
| Specificity | 90.00 | +LR | 1.82 |
| Sensitivity | 29.25 | 95% CI | 1.3-2.7 |
| 95% Confidence Interval | 17.92-41.51 | −LR | 0.65 |
| Criterion | ≤0.032746075 | 95% CI | 0.5-0.8 |

miR-145a-5p

| Area Under the ROC Curve | 0.623 | Criterion | ≤0.108623657 |
|---|---|---|---|
| (AUC) | | | |
| Standard Error | 0.0413 | Sensitivity | 50.00 |
| 95% Confidence Interval | 0.549-0.693 | 95% CI | 40.1-59.9 |
| Significance Level P | 0.0029 | Specificity | 71.25 |
| (Area = 0.5) | | | |
| Estimated sensitivity at fixed specificity | | 95% CI | 60.0-80.8 |
| Specificity | 90.00 | +LR | 1.74 |
| Sensitivity | 18.87 | 95% CI | 1.2-2.6 |
| 95% Confidence Interval | 8.49-33.02 | −LR | 0.70 |
| Criterion | ≤0.049521 | 95% CI | 0.6-0.9 |

miR-146a-5p

| Area Under the ROC Curve | 0.628 | Criterion | ≤1.279229286 |
|---|---|---|---|
| (AUC) | | | |
| Standard Error | 0.0413 | Sensitivity | 71.70 |
| 95% Confidence Interval | 0.554-0.698 | 95% CI | 62.1-80.0 |
| Significance Level P | 0.0019 | Specificity | 48.75 |
| (Area = 0.5) | | | |
| Estimated sensitivity at fixed specificity | | 95% CI | 37.4-60.2 |
| Specificity | 90.00 | +LR | 1.40 |
| Sensitivity | 22.64 | 95% CI | 1.1-1.8 |
| 95% Confidence Interval | 6.60-36.79 | −LR | 0.58 |
| Criterion | ≤0.304521943 | 95% CI | 0.4-0.8 |

miR-155a-5p

| Area Under the ROC Curve | 0.671 | Criterion | ≤0.891603925 |
|---|---|---|---|
| (AUC) | | | |
| Standard Error | 0.0404 | Sensitivity | 87.74 |
| 95% Confidence Interval | 0.598-0.738 | 95% CI | 79.9-93.3 |
| Significance Level P | <0.0001 | Specificity | 41.25 |
| (Area = 0.5) | | | |
| Estimated sensitivity at fixed specificity | | 95% CI | 30.4-52.8 |
| Specificity | 90.00 | +LR | 1.49 |
| Sensitivity | 22.64 | 95% CI | 1.2-1.8 |
| 95% Confidence Interval | 13.21-45.14 | −LR | 0.30 |
| Criterion | ≤0.194107866 | 95% CI | 0.2-0.5 |

miR-210-3p

| Area Under the ROC Curve | 0.719 | Criterion | ≤0.167944775 |
|---|---|---|---|
| (AUC) | | | |
| Standard Error | 0.0378 | Sensitivity | 92.45 |
| 95% Confidence Interval | 0.649-0.782 | 95% CI | 85.7-96.7 |
| Significance Level P | <0.0001 | Specificity | 42.50 |
| (Area = 0.5) | | | |
| Estimated sensitivity at fixed specificity | | 95% CI | 31.5-54.1 |
| Specificity | 90.00 | +LR | 1.61 |
| Sensitivity | 34.91 | 95% CI | 1.3-2.0 |
| 95% Confidence Interval | 23.69-46.23 | −LR | 0.18 |
| Criterion | ≤0.041728 | 95% CI | 0.09-0.4 |

miR-342-3p

| Area Under the ROC Curve | 0.644 | Criterion | ≤3.281807058 |
|---|---|---|---|
| (AUC) | | | |
| Standard Error | 0.0409 | Sensitivity | 81.13 |
| 95% Confidence Interval | 0.570-0.712 | 95% CI | 72.4-88.1 |
| Significance Level P | 0.0004 | Specificity | 43.75 |
| (Area = 0.5) | | | |
| Estimated sensitivity at fixed specificity | | 95% CI | 32.7-55.3 |
| Specificity | 90.00 | +LR | 1.44 |
| Sensitivity | 25.47 | 95% CI | 1.2-1.8 |
| 95% Confidence Interval | 10.30-38.68 | −LR | 0.43 |
| Criterion | ≤0.903942535 | 95% CI | 0.3-0.7 |

CI = Confidence Interval
LR = Likelihood Ratio

### Example 7

Example 7 describes a successful detection of CHH using selected miRNA markers in a selected sample of patients.

29 monitored patients have CHH. Blood samples of these selected patients are analyzed following the procedure described in Example 1 and levels of selected 4 miRNAs are determined (miR-1-3p, miR-20a-5p, miR-126-3p, miR-146a-5p). Up-regulation of these selected miRNA biomarkers, whose levels exceed the minimum values determined by a statistical analysis for a 10% FPR, is observed in 23 of 29 patients, which corresponds to a successful detection in 79.3% of cases. Statistical analysis of the obtained data provides the following values of specificity, sensitivity, 95% CI, and criterion:

Combined screening of CHH (4 selected miRNA)

| Area Under the ROC Curve | 0.880 | Criterion | >0.298082817 |
|---|---|---|---|
| (AUC) | | | |
| Standard Error | 0.0421 | Sensitivity | 79.31 |
| 95% Confidence Interval | 0.804-0.935 | 95% CI | 60.3-92.0 |
| Significance Level P | <0.0001 | Specificity | 91.25 |
| (Area = 0.5) | | | |
| Estimated sensitivity at fixed specificity | | 95% CI | 82.8-96.4 |
| Specificity | 90.00 | +LR | 9.06 |
| Sensitivity | 79.31 | 95% CI | 4.4-18.8 |
| 95% Confidence Interval | 62.07-93.10 | −LR | 0.23 |
| Criterion | >0.255337049 | 95% CI | 0.1-0.5 |

miR-1-3p

| Area Under the ROC Curve | 0.823 | Criterion | >0.199151 |
|---|---|---|---|
| (AUC) | | | |
| Standard Error | 0.0507 | Sensitivity | 79.31 |
| 95% Confidence Interval | 0.739-0.890 | 95% CI | 60.3-92.0 |
| Significance Level P | <0.0001 | Specificity | 76.25 |
| (Area = 0.5) | | | |
| Estimated sensitivity at fixed specificity | | 95% CI | 65.4-85.1 |
| Specificity | 90.00 | +LR | 3.34 |
| Sensitivity | 51.72 | 95% CI | 2.2-5.2 |
| 95% Confidence Interval | 29.63-72.41 | −LR | 0.27 |
| Criterion | >0.377841 | 95% CI | 0.1-0.6 |

miR-20a-5p

| Area Under the ROC Curve | 0.820 | Criterion | >2.302443 |
|---|---|---|---|
| (AUC) | | | |
| Standard Error | 0.0410 | Sensitivity | 82.76 |
| 95% Confidence Interval | 0.735-0.887 | 95% CI | 64.2-94.2 |
| Significance Level P | <0.0001 | Specificity | 70.00 |
| (Area = 0.5) | | | |
| Estimated sensitivity at fixed specificity | | 95% CI | 58.7-79.7 |
| Specificity | 90.00 | +LR | 2.76 |
| Sensitivity | 44.83 | 95% CI | 1.9-4.0 |
| 95% Confidence Interval | 27.59-66.90 | −LR | 0.25 |
| Criterion | >3.587078 | 95% CI | 0.1-0.6 |

miR-126-3p

| Area Under the ROC Curve | 0.691 | Criterion | >0.189274 |
|---|---|---|---|
| (AUC) | | | |
| Standard Error | 0.0519 | Sensitivity | 100.00 |
| 95% Confidence Interval | 0.595-0.776 | 95% CI | 88.1-100.0 |
| Significance Level P | 0.0002 | Specificity | 36.25 |
| (Area = 0.5) | | | |
| Estimated sensitivity at fixed specificity | | 95% CI | 25.8-47.8 |
| Specificity | 90.00 | +LR | 1.57 |
| Sensitivity | 20.69 | 95% CI | 1.3-1.9 |
| 95% Confidence Interval | 1.74-44.83 | −LR | 0.00 |
| Criterion | >0.659038 | 95% CI | |

miR-146-5p

| Area Under the ROC Curve | 0.857 | Criterion | >2.073306 |
|---|---|---|---|
| (AUC) | | | |
| Standard Error | 0.0400 | Sensitivity | 79.31 |
| 95% Confidence Interval | 0.777-0.917 | 95% CI | 60.3-92.0 |
| Significance Level P | <0.0001 | Specificity | 82.50 |
| (Area = 0.5) | | | |
| Estimated sensitivity at fixed specificity | | 95% CI | 72.4-90.1 |
| Specificity | 90.00 | +LR | 4.53 |
| Sensitivity | 62.52 | 95% CI | 2.7-7.6 |
| 95% Confidence Interval | 27.59-89.66 | LR | 0.25 |
| Criterion | >2.744534 | 95% CI | 0.1-0.5 |

CI = Confidence Interval
LR = Likelihood Ratio

### Example 8

Example 8 describes a successful prediction of SGA using selected miRNA markers in a selected sample of patients.

Fetuses of 37 monitored patients are diagnosed with SGA during pregnancy and these patients give birth between the 33rd and the 40th gestational week, with 4 patients giving birth before the 34th gestational week (early SGA) and 33 give birth after the 34th gestational week (late SGA). Blood samples of these selected patients are analyzed following the procedure described in Example 1 and levels of selected 8 miRNAs are determined (miR-1-3p, miR-20a-5p, miR-20b-5p, miR-126-3p, miR-130b-3p, miR-146a-5p, miR-181a-5p, miR-499a-5p). Up-regulation of these selected miRNA biomarkers, whose levels exceed the minimum values determined by a statistical analysis for a 10% FPR, is observed in 31 of 37 patients (1 early SGA, 30 late SGA), which corresponds to a successful prediction in 83.8% of cases. Statistical analysis of the obtained data provides the following values of specificity, sensitivity, 95% CI, and criterion:

Combined screening of SGA (8 selected miRNA)

| Area Under the ROC Curve | 0.926 | Criterion | >0.613361244 |
|---|---|---|---|
| (AUC) | | | |
| Standard Error | 0.0287 | Sensitivity | 75.68 |
| 95% Confidence Interval | 0.863-0.966 | 95% CI | 58.8-88.2 |
| Significance Level P | <0.0001 | Specificity | 100.00 |
| (Area = 0.5) | | | |
| Estimated sensitivity at fixed specificity | | 95% CI | 95.5-100.0 |
| Specificity | 90.00 | +LR | |
| Sensitivity | 83.78 | 95% CI | |
| 95% Confidence Interval | 70.27-94.59 | −LR | 0.24 |
| Criterion | >0.272302152 | 95% CI | 0.1-0.4 |

miR-1-3p

| Area Under the ROC Curve | 0.790 | Criterion | >0.23812 |
|---|---|---|---|
| (AUC) | | | |
| Standard Error | 0.0489 | Sensitivity | 70.27 |
| 95% Confidence Interval | 0.705-0.860 | 95% CI | 53.0-84.1 |
| Significance Level P | <0.0001 | Specificity | 85.00 |
| (Area = 0.5) | | | |
| Estimated sensitivity at fixed specificity | | 95% CI | 75.3-92.0 |
| Specificity | 90.00 | +LR | 4.68 |
| Sensitivity | 56.76 | 95% CI | 2.7-8.2 |
| 95% Confidence Interval | 32.43-75.68 | −LR | 0.35 |
| Criterion | >0.377841 | 95% CI | 0.2-0.6 |

miR-20a-5p

| Area Under the ROC Curve | 0.842 | Criterion | >2.413833 |
|---|---|---|---|
| (AUC) | | | |
| Standard Error | 0.0408 | Sensitivity | 81.08 |
| 95% Confidence Interval | 0.763-0.902 | 95% CI | 64.8-92.0 |
| Significance Level P | <0.0001 | Specificity | 76.25 |
| (Area = 0.5) | | | |
| Estimated sensitivity at fixed specificity | | 95% CI | 65.4-85.1 |
| Specificity | 90.00 | +LR | 3.41 |
| Sensitivity | 59.46 | 95% CI | 2.2-5.2 |
| 95% Confidence Interval | 29.73-78.38 | −LR | 0.25 |
| Criterion | >3.587078 | 95% CI | 0.1-0.5 |

miR-20b-5p

| Area Under the ROC Curve | 0.715 | Criterion | >2.281479 |
|---|---|---|---|
| (AUC) | | | |
| Standard Error | 0.0484 | Sensitivity | 70.27 |
| 95% Confidence Interval | 0.624-0.794 | 95% CI | 53.0-84.1 |
| Significance Level P | <0.0001 | Specificity | 65.00 |
| (Area = 0.5) | | | |
| Estimated sensitivity at fixed specificity | | 95% CI | 53.5-75.3 |
| Specificity | 90.00 | +LR | 2.01 |
| Sensitivity | 21.62 | 95% CI | 1.4-2.9 |
| 95% Confidence Interval | 2.70-48.65 | −LR | 0.46 |
| Criterion | >4.517659 | 95% CI | 0.3-0.8 |

miR-126-3p

| Area Under the ROC Curve | 0.695 | Criterion | >0.376441 |
|---|---|---|---|
| (AUC) | | | |
| Standard Error | 0.0494 | Sensitivity | 62.16 |
| 95% Confidence Interval | 0.603-0.777 | 95% CI | 44.8-77.5 |
| Significance Level P | 0.0001 | Specificity | 71.25 |
| (Area = 0.5) | | | |
| Estimated sensitivity at fixed specificity | | 95% CI | 60.0-80.8 |
| Specificity | 90.00 | +LR | 2.16 |
| Sensitivity | 21.62 | 95% CI | 1.4-3.3 |
| 95% Confidence Interval | 2.70-56.76 | −LR | 0.53 |
| Criterion | >0.659038 | 95% CI | 0.3-0.8 |

miR-130b-3p

| Area Under the ROC Curve | 0.665 | Criterion | >1.14816 |
|---|---|---|---|
| (AUC) | | | |
| Standard Error | 0.0540 | Sensitivity | 56.76 |
| 95% Confidence Interval | 0.572-0.750 | 95% CI | 39.5-72.9 |
| Significance Level P | 0.0022 | Specificity | 75.00 |
| (Area = 0.5) | | | |
| Estimated sensitivity at fixed specificity | | 95% CI | 64.1-84.0 |
| Specificity | 90.00 | +LR | 2.27 |
| Sensitivity | 18.92 | 95% CI | 1.4-3.6 |
| 95% Confidence Interval | 2.70-45.95 | −LR | 0.58 |
| Criterion | >1.856532 | 95% CI | 0.4-0.9 |

miR-146a-5p

| Area Under the ROC Curve | 0.859 | Criterion | >2.073306 |
|---|---|---|---|
| (AUC) | | | |
| Standard Error | 0.0382 | Sensitivity | 78.38 |
| 95% Confidence Interval | 0.783-0.917 | 95% CI | 61.8-90.2 |
| Significance Level P | <0.0001 | Specificity | 82.50 |
| (Area = 0.5) | | | |
| Estimated sensitivity at fixed specificity | | 95% CI | 72.4-90.1 |
| Specificity | 90.00 | +LR | 4.48 |
| Sensitivity | 64.86 | 95% CI | 2.7-7.4 |
| 95% Confidence Interval | 37.84-83.78 | −LR | 0.26 |
| Criterion | >2.744534 | 95% CI | 0.1-0.5 |

miR-181a-5p

| Area Under the ROC Curve | 0.757 | Criterion | >0.201449 |
|---|---|---|---|
| (AUC) | | | |
| Standard Error | 0.0467 | Sensitivity | 91.89 |
| 95% Confidence Interval | 0.669-0.831 | 95% CI | 78.1-98.3 |
| Significance Level P | <0.0001 | Specificity | 55.00 |
| (Area = 0.5) | | | |
| Estimated sensitivity at fixed specificity | | 95% CI | 43.5-66.2 |
| Specificity | 90.00 | +LR | 2.04 |
| Sensitivity | 32.43 | 95% CI | 1.6-2.6 |
| 95% Confidence Interval | 16.22-56.76 | −LR | 0.15 |
| Criterion | >0.521629 | 95% CI | 0.05-0.4 |

miR-499a-5p

| Area Under the ROC Curve | 0.663 | Criterion | >0.299081 |
|---|---|---|---|
| (AUC) | | | |
| Standard Error | 0.0514 | Sensitivity | 75.68 |
| 95% Confidence Interval | 0.570-0.748 | 95% CI | 58.8-88.2 |
| Significance Level P | 0.0015 | Specificity | 53.75 |
| (Area = 0.5) | | | |
| Estimated sensitivity at fixed specificity | | 95% CI | 42.2-65.0 |
| Specificity | 90.00 | +LR | 1.64 |
| Sensitivity | 21.62 | 95% CI | 1.2-2.2 |
| 95% Confidence Interval | 5.41-39.31 | −LR | 0.45 |
| Criterion | >1.191424 | 95% CI | 0.2-0.8 |

CI = Confidence Interval
LR = Likelihood Ratio

### Example 9

Example 9 describes a successful prediction of GH using a selected miRNA marker in a selected sample of patients.

83 monitored patients are diagnosed with GH during pregnancy. Blood samples of these selected patients are analyzed following the procedure described in Example 1 and levels of the selected miRNA are determined (miR-181a-5p). Up-regulation of this selected miRNA biomarker, whose level exceeds the minimum value determined by a statistical analysis for a 10% FPR, is observed in 19 of 83 patients, which corresponds to a successful prediction in 22.9% of cases. Statistical analysis of the obtained data provides the following values of specificity, sensitivity, 95% CI, and criterion:

Screening of GH (miR-181a-5p)

| Area Under the ROC Curve | 0.649 | Criterion | >0.261811 |
|---|---|---|---|
| (AUC) | | | |
| Standard Error | 0.0429 | Sensitivity | 61.45 |
| 95% Confidence Interval | 0.570-0.722 | 95% CI | 50.1-71.9 |
| Significance Level P (Area = 0.5) | 0.0005 | Specificity | 65.00 |
| Estimated sensitivity at fixed specificity | | 95% CI | 53.5-75.3 |
| Specificity | 90.00 | +LR | 1.76 |
| Sensitivity | 22.89 | 95% CI | 1.2-2.5 |
| 95% Confidence Interval | 12.18-36.14 | LR | 0.59 |
| Criterion | >0.521629 | 95% CI | 0.4-0.8 |

CI = Confidence Interval
LR = Likelihood Ratio

### INDUSTRIAL APPLICABILITY

Method of prediction of pregnancy complications based on the expression profile of cardiovascular miRNAs is industrially applicable in the clinical practice of gynecology and obstetrics in the laboratory analysis of collected samples of biological material.

1. Method of prediction of pregnancy complications in the form of preeclampsia, fetal growth restriction, gestational hypertension, small for gestational age fetus, and preterm birth and detection of chronic hypertension, characterized in that pregnant women are screened to determine the expression profile of one or more miRNAs in whole peripheral venous blood collected between the 10th and the 13th gestational week, whereas said one or more miRNAs are selected from the group miR-1-3p to determine its up-regulation, miR-16-5p to determine its up-regulation or down-regulation, miR-17-5p to determine its up-regulation, miR-20a-5p to determine its up-regulation, miR-20b-5p to determine its up-regulation or down-regulation, miR-21-5p to determine its up-regulation or down-regulation, miR-24-3p to determine its down-regulation, miR-26a-5p to determine its down-regulation, miR-92a-3p to determine its down-regulation, miR-100-5p to determine its up-regulation, miR-103a-3p to determine its up-regulation, miR-126-3p to determine its up-regulation or down-regulation, miR-130b-3p to determine its up-regulation, miR-133a-3p to determine its down-regulation, miR-143-3p to determine its up-regulation, miR-145-5p to determine its up-regulation or down-regulation, miR-146a-5p to determine its up-regulation or down-regulation, miR-155-5p to determine its up-regulation or down-regulation, miR-181a-5p to determine its up-regulation, miR-195-5p to determine its up-regulation, miR-210-3p to determine its down-regulation, miR-221-3p to determine its down-regulation, miR-342-3p to determine its up-regulation or down-regulation, miR-499a-5p to determine its up-regulation, and miR-574-3p to determine its up-regulation or down-regulation.

2. Method according to claim 1, characterized in that the screened miRNAs for prediction of preeclampsia are up-regulated miR-20a-5p, up-regulated miR-21-5p, up-regulated miR-126-3p, up-regulated miR-143-3p, up-regulated miR-145-5p, up-regulated miR-146a-5p, up-regulated miR-181a-5p, and up-regulated miR-574-3p.

3. Method according to claim 1, characterized in that the screened miRNAs for prediction of fetal growth restriction are up-regulated miR-16-5p, up-regulated miR-20a-5p, up-regulated miR-100-5p, up-regulated miR-143-3p, up-regulated miR-145-5p, up-regulated miR-146a-5p, up-regulated miR-181a-5p, up-regulated miR-195-5p, up-regulated miR-342-3p, and up-regulated miR-574-3p.

4. Method according to claim 1, characterized in that the screened miRNA for prediction of gestational hypertension is up-regulated miR-181a-5p.

5. Method according to claim 1, characterized in that the screened miRNAs for prediction of small for gestational age fetus are up-regulated miR-1-3p, up-regulated miR-20a-5p, up-regulated miR-20b-5p, up-regulated miR-126-3p, up-regulated miR-130b-3p, up-regulated miR-146a-5p, up-regulated miR-181a-5p, and up-regulated miR-499a-5p.

6. Method according to claim 1, characterized in that the screened miRNAs for prediction of preterm birth are down-regulated miR-16-5p, down-regulated miR-20b-5p, down-regulated miR-21-5p, down-regulated miR-24-3p, down-regulated miR-26a-5p, down-regulated miR-92a-3p, down-regulated miR-133a-3p, down-regulated miR-145-5p, down-regulated miR-146a-5p, down-regulated miR-155-5p, down-regulated miR-210-3p, and down-regulated miR-342-3p.

7. Method according to claim 6, characterized in that the preterm birth is in the form of spontaneous preterm birth or preterm premature rupture of membranes.

8. Method according to claim 1, characterized in that the screened miRNAs for detection of chronic hypertension are up-regulated miR-1-3p, up-regulated miR-20a-5p, up-regulated miR-126-3p, and up-regulated miR-146a-5p.
